(19) United States
(12) Patent Application Publication
(10) Pub. No.: US 2004/0014772 A1
(43) Pub. Date: Jan. 22, 2004

(54) Compositions and methods for treating glomerular disorders

(76) Inventors: Sudhir V. Shah, Little Rock, AR (US); Radhakrishna Baliga, Madison, MS (US)

Correspondence Address: Gilbreth & Associates, P.C., P.O. Box 2428, Bellaire, TX, 77402-2428, US

(21) Appl. No.: 10/385,416

(22) Filed: Mar. 10, 2003

Related U.S. Application Data
(63) Continuation of application No. 09/672,296, filed on Mar. 29, 2000, now abandoned.

(51) Int. Cl. A61K 31/495; A61K 31/426; A61K 31/353; A61K 31/325

(52) U.S. Cl. 514/255.04; 514/456; 514/365; 514/478

(57) ABSTRACT

Compositions and methods for treating a glomerular disorder. Compositions and methods comprise a cytochrome P450 inhibitor. Preferred inhibitors inhibit a cytochrome P450 2B family member. Compositions and methods reduce proteinuria associated with a glomerular disorder, such as, minimal change nephrotic syndrome.

### RELATED APPLICATION DATA

This application is a continuation of and claims priority to U.S. application Ser. No. 09/672,296, filed Mar. 29, 2000, entitled “Compositions and methods for treating glomerular disorders.”

### REFERENCE TO GOVERNMENT FUNDING

This work was supported by Project Grant KCF98029 from Kidney Care, Inc. Foundation.

### BACKGROUND OF THE INVENTION

1. Field of the Invention

The present invention relates to nephrotic disorders, and to compositions and methods of treating nephrotic disorders. In another aspect, the present invention relates to glomerular disorders, and to compositions and methods for treating glomerular disorders. In even another aspect, the present invention is related to compositions comprising an agent that inhibits a member of the cytochrome P-450 family, and to methods for using such compositions to treat glomerulonephritides. In yet another aspect, the present invention relates to nephrotic syndromes, to compositions comprising an agent that inhibits a cytochrome P450, and to methods of using same to treat nephrotic syndromes. In even yet another aspect, the present invention relates to compositions comprising a cytochrome P-450 inhibitor, and to methods of using such compositions to treat minimal change nephrotic syndrome.

2. Description of the Related Art

Minimal change nephrotic syndrome (MCNS) is the most common nephrotic syndrome affecting children between 2 to 6 years of age, and accounts for more than 75% of the cases of nephrotic syndrome in children. In adults, MCNS accounts for about 30% of nephrotic syndrome. Unlike acute renal failure which affects the tubules, MCNS is a disease of the glomeruli, which function as filtering units of the kidneys. As is the case with many other glomerulonephritides, a hallmark of MCNS is proteinuria.

Current methods for treating individuals afflicted with glomerulonephritides, such as, MCNS, are empirical and consist mostly of corticosteroids and, at times, immunosuppressive agents. These traditional MCNS treatments are limited by their adverse side effects such as, growth failure, cushingoid features and bone deformities.

For acute renal failure, it has been suggested that the iron storage proteins ferritin, transferrin, iron-rich mitochondria, and extracellular heme proteins, such as hemoglobin and myoglobin, might serve as sources of catalytic iron. However, the actual sources of iron that catalyze free radical reactions in acute renal failure remains an actively researched area.

For example, Paller et al., 1994, PNAS 91:70002-70006, disclose the hypothesis that hydroxyl radical formation during reoxygenation of the kidney is mediated by cytochrome P-450.

Ueda et al, 1993, Am. L. Physiol 265:F435-F439 disclose that isolated renal cortical mitochondria release iron when exposed to the nephrotoxic drug gentamicin.

Baliga et al, 1996, Kidney International 49:362-369, disclose evidence for cytochrome P-450 as a source of catalytic iron in a model of myoglobinuric acute renal failure induced by glycerol, wherein two cytochrome P450 inhibitors tested therein provided functional and histological protection against the acute renal failure.

Baliga et al., 1996, Kidney International 50:1118-1124, disclose a role of cytochrome P-450 in hydrogen peroxide-induced cytotoxicity in LLC-PK1 cells, wherein two cytochrome P450 inhibitors tested therein provided beneficial effects against hydrogen peroxide-induced cell injury.

Zager et al., 1997, Kidney International 51:728-738, suggest that the formation of iron/H₂O₂-based reactive intermediates in mitochondria may be responsible for the cell damage in an in vitro model of myoglobin cytotoxicity.

Baliga et al., 1998, Kidney International 53:394-401, disclose a hypothesis suggesting a role for iron in mediating tissue injury via hydroxy radicals in a cisplatin-induced model of nephrotoxicity.

Baliga et al., November 1998, Kidney International 54(5), 1562-1569, disclose a role for cytochrome P450 as a source of catalytic iron in cisplatin-induced nephrotoxicity.

Ueda et al, 1996, Kidney International 49:370-373, and Ueda et al, 1997, Kidney: A Current Survey of World Literature 6:143-146, disclose a pathogenic role for reactive oxygen metabolites (ROMs) in glomerular disease, a source of the glomerular catalytic iron is not disclosed. Thus, while ROMs are believed to be important in mediating renal injury, the molecular mechanisms underlying the activation of a catalytic iron source in glomerular disease, such as MCNS, are not known.

However, unlike models for acute renal failure, a role for a CYP in a glomerular disease or disease model, such as, puromycin aminonucleoside (PAN)-induced MCNS, has not previously been disclosed.

Despite the advances made in the field of glomerular disease mechanisms, there remains a need for treatments for individuals having a glomerular disorder wherein the composition comprises an inhibitor of a cytochrome P450. There is another need in the art for compositions and methods for treating an individual having a glomerular disorder, wherein the compositions and methods lack the adverse side effects associated with traditional therapies, such as, steroids and immunosuppressants. There is even another need in the art for compositions and methods for treating a patient having a proteinuric disorder, wherein the compositions comprise an agent that inhibits a cytochrome P450. There is even another need in the art for compositions and methods for treating a patient having a proteinuric disorder, such as minimal change nephrotic syndrome, wherein the compositions comprise an agent which inhibits a cytochrome P450 superfamily member.

### SUMMARY OF THE INVENTION

It is an object of the present invention to provide compositions and methods for treating an individual having a glomerular disorder, wherein the compositions and methods lack the adverse side effects associated with traditional therapies, such as, steroids and immunosuppressant.

It is even another object of the present invention to provide for compositions and methods for reducing the proteinuria associated with a glomerular disorder in an individual afflicted with a glomerular disease.

It is still another object of the present invention to provide for compositions and methods for treating an individual having a glomerular disorder wherein the compositions and methods comprise an agent that inhibits a cytochrome P450 superfamily member.

These and other objects of the present invention will become apparent to those of skill in the art upon review of this specification, including its drawings and claims.

One embodiment of the present invention is directed to a method for treating a patient, the method comprising the step of: (a) administering a therapeutically effective amount of a composition to a patient having a glomerular disorder, wherein the composition comprises an effective amount of an agent that inhibits a glomerular cytochrome P450.

Another embodiment of the present invention is directed to A method for treating a patient, the method comprising the step of: (a) administering a therapeutically effective amount of a composition to a patient having a glomerular disorder, wherein said composition comprises an effective amount of an expression vector comprising a sequence encoding an inhibitor of a cytochrome P450.

Even another embodiment of the present invention is directed to a method for treating a patient, the method comprising the step of: (a) administering a therapeutically effective amount of a composition to a patient, wherein said composition comprises an agent that inhibits a cytochrome P450 superfamily member, and wherein said patient exhibits a urinary protein excretion value of greater than about 300 mg in a twenty four hour time period.

Still another embodiment of the present invention is directed to a method for treating a patient, the method comprising the step of: (a) administering a therapeutically effective amount of a composition to a patient, wherein said composition comprises an expression vector comprising a sequence encoding a cytochrome P450 inhibitor, and wherein said patient has a urinary protein excretion value of greater than about 300 mg per twenty-four hour time period

Yet another embodiment of the present invention is directed to a method for treating a patient, the method comprising the step of: (a) administering a therapeutically effective amount of a composition to a patient, wherein said composition comprises an agent that inhibits a cytochrome P450 superfamily member, and wherein said agent does not inhibit kidney function.

### BRIEF DESCRIPTION OF THE DRAWINGS

FIG. 1A shows the levels of proteinuria in PAN treated animals. A PAN-induced model of minimal change nephrotic syndrome was induced by a single intravenous injection of PAN (7.5 mg/100 g BW) to rats. The animals were sacrificed 7 days after PAN injection. Values are means±SE. *p<0.01 comparing PAN-treated rats with control animals.

FIG. 1B shows the bleomycin detectable iron content levels in the glomeruli of PAN treated animals. A PAN-induced model of minimal change nephrotic syndrome was induced by a single intravenous injection of PAN (7.5 mg/100 g BW) to rats. The animals were sacrificed 7 days after PAN injection. Values are means±SE. *p<0.01 comparing PAN-treated rats with control animals.

FIG. 2A shows cytochrome P450 content in kidney in PAN-induced nephrotic syndrome in rats. Values are means±SE.

FIG. 2B shows cytochrome P450 content in the liver in PAN-induced nephrotic syndrome in rats. Values are means±SE.

FIG. 3 shows the effect of cytochrome P450 inhibitors on the bleomycin detectable iron content in the glomeruli of PAN-treated rats. Values are means±SE. *p<0.05 compared with control rats. ⁺p<0.05 compared with PAN treatment alone.

FIG. 4 shows the effect of cytochrome P450 inhibitors on cytochrome P450 content in the glomeruli of PAN-treated rats. Values are means±SE.

FIG. 5 shows the effect of cytochrome P450 inhibitors on proteinuria in PAN-treated rats. Values are means±SE. *p<0.05 compared with the rats treated with PAN alone.

FIG. 6 shows the effect of cytochrome P450 inhibitors on serum albumin in rats treated with PAN. Values are means±SE. *p<0.05 compared with the control rats.

FIG. 7A shows renal function as measured by serum creatinine in rats injected with PAN with or without cytochrome P450 inhibitors. Values are means±SE. *p<0.05 compared with the control rats.

FIG. 7B shows renal function as measured by blood urea nitrogen in rats injected with PAN with or without cytochrome P450 inhibitors. Values are means±SE. *p<0.05 compared with the control rats.

FIG. 8 shows the effect of cytochrome P450 inhibitors on the release of catalytic iron in glomerular epithelial cells GEC exposed to PAN. Confluent GEC were incubated with a cytotoxic dose of PAN (1.5 mM) for a period of time less than the time at which substantial cell killing occurs (1 h). Cytochrome P450 inhibitors PB (25 μM), CM (2 mM) and RN (1 mM, as a control for CM) were preincubated with GEC for 30-60 min and then washed with chelexed HBSS followed by addition of PAN. Values are means±SE. *p<0.05 compared with the control; ⁺p<0.05 compared with GEC exposed to PAN alone.

FIG. 9 shows the effect of cytochrome P450 inhibitors on hydroxyl radical generation in GEC exposed to PAN. Confluent GEC were incubated with cytotoxic dose of PAN (1.5 mM) for a period of time less than the time at which substantial cell killing occurs (1h). Cytochrome P450 inhibitors PB (25 μM), CM (2 mM) and RN (1 mM, as a control for CM) were preincubated with GEC for 30-60 min and then washed with HBSS followed by addition of PAN. Values are means±SE. *p<0.05 compared with the control; +p<0.05 compared with PAN alone.

FIG. 10A shows the concentration-dependent effect of PAN (0 to 2 mM for 48h) on cytotoxicity to GEC as measured by trypan blue exclusion assay. Values are means±SE. N=2.

FIG. 10B shows the time-dependent effect of PAN (0 to 4 days at PAN dose of 1.5 mM)on cytotoxicity in GEC as measured by trypan blue exclusion assay. Values are means±SE. N=2.

FIG. 11 shows the effect of cytochrome P450 inhibitors on GEC cell death in GEC exposed to PAN. Confluent GEC were incubated with a cytotoxic dose of PAN (1.5 mM) for a period of time necessary to induce consistent cytotoxicity (48 h). Cytochrome P450 inhibitors PB (25 μM), CM (2 mM) and RN (1 mM, as a control for CM) were preincubated with GEC for 30-60 min and then washed with HBSS followed by addition of PAN. Values are means±SE. *p<0.05 compared with GEC exposed to PAN alone at concentration indicated in the figure. N=3.

FIGS. 12A and 12B provide the immunohistochemistry and western blot data, respectively, of the identification and localization of CYP2B1 in the rat kidney. FIG. 12C provides data showing CYP2B1 is present in the glomeruli but not in the tubules of the kidney.

FIG. 13A shows the effect of CYP450 inhibitors on the proteinuria (mg /24h) in rats treated with puromycin aminonucleoside (PAN). Symbols are: ●=control, N=6; [symbol] =PAN, N=10; ▾=CM, N=6; ▪=PIP, N=8. FIG. 13B shows the effect of CYP inhibitors on the serum albumin (g/dL) in PAN-treated rats. Values are means±SE, N=6 to 10. *=P<0.05 compared with the rats treated with PAN alone. +=P<0.05 compared with control rats.

FIGS. 14A-14E provide data from a histochemical cerium-H₂O₂ precipitation method in rat kidney 6 hours and 4 days after PAN injection.

FIGS. 15A and 15B show the effect of CYP inhibitors on the bleomycin detectable iron content in the glomeruli of PAN-treated rats at 6 hours, on day 4, and on day 7. Values are means±SE, N=3 to 5, *=P<0.05 compared with control rats, +=P<0.05 compared with rats treated with PAN alone.

FIG. 16 provides the immunohistochemistry data for CYP2B1 (left column), heme oxidase (HO-1) (middle column), and ferritin (right column) following PAN treatment +/−piperine, +/−cimetidine, at 6 hours and at day 4.

FIG. 17 provides western blot analysis of CYP2B1 protein in the glomeruli of rats injected with PAN +/−administration of CM and PIP. The blots are representative of results from 6 to 10 animals per experimental group, 6 hours after and 7 days after PAN injection.

FIG. 18 provides western blot analysis of heme oxidase (HO-1) protein in the glomeruli of rats injected with PAN +/−administration of CM and PIP. The blots are representative of results from 6 to 10 animals per experimental group, 6 hours after and 7 days after PAN injection.

FIGS. 19A and 19B show the induction of HO-1 mRNA in the kidney of PAN-treated rats and the effect of CYP2B1 inhibitor PIP on this induction.

FIGS. 20a and 20B show the localization of CYP2B1 in GEC by immunohistochemistry and western blot analysis, respectively.

FIGS. 21A and 21B respectively show the concentration dependent and time dependent cytotoxicity of PAN in GEC.

FIGS. 22A, 22B and 22C show the effect of piperine and cimetidine on the cytotoxicity of treatment of GEC with 1.5 mM PAN for 48 hr at 37° C., as measured by trypan blue exclusion assay (21A and 21B) and LDH release (21C).

FIG. 23A is a time course of H₂O₂ generation in GEC exposed to 1.5 mM PAN; FIG. 23B is a western blot analysis of CYP2B1 content in control GEC and PAN-treated GEC (1.5 mM for 2 hr at 37° C.); and FIG. 22C is a time course of catalytic iron release (nmol/mg protein) from GEC exposed to PAN.

FIG. 24 shows the effect of the CYP inhibitors piperine and cimetidine on H₂O₂ generation in GEC exposed to 1.5 mM PAN.

FIG. 25 is a western blot analysis of CYP2B1 protein in the microsome of GEC exposed to PAN (1.5 mM for 2 hr at 37° C.)+/−the H₂O₂ scavenger pyruvate (10 mM) and the CYP inhibitors CM (2 mM) and PIP (0.2 mM). The blot shown is representative of the results from 3 groups of studies.

FIG. 26 shows the effect of PIP (0.2 mM) and CM (2 mM) on iron release from GEC treated with PAN (1.5 mM for 1 hr at 37° C.). Values are means±SE, N=3 to 5, *=P<0.05 compared to control, +=P<0.05 compared to GEC exposed to PAN alone.

FIGS. 27A and 27B respectively show the hydrogen peroxide generation, and catalytic iron release from microsomes isolated from GEC exposed to PAN. Values are means±SE, N=4, *=P<0.05 compared to control.

FIG. 28 shows the effect of the H₂O₂ scavenger pyruvate (10 mM) and the CYP inhibitors CM (2 mM) and PIP (0.2 mM) on hydroxyl radical formation in GEC treated with PAN. Values are means±SE, N=3, *=P<0. 05 compared to control, +=P<0.05 compared to GEC exposed to PAN alone.

FIG. 29 is a western blot analysis of HO-1 induction in GEC exposed to PAN +/−the CYP inhibitors PIP (0.2 mM), CM (2 mM) and the H₂O₂ scavenger pyruvate (10 mM). The blot shown is representative of the results from 3 groups of studies.

### DETAILED DESCRIPTION OF THE INVENTION

The present invention is directed to compositions and methods for treating a glomerular disorder. The compositions and methods of the present invention are advantageous over traditional treatments for glomerular disorders in that they are not associated with adverse side effects, yet are effective in reducing proteinuria.

As used herein, the phrase “glomerular disorder” includes any glomerular disease and/or any glomerular injury such as, for example, minimal change disease, focal segmental sclerosis, membranous nephropathy, diabetic nephropathy, amyloidosis, and proliferative glomerular nephritis such as, anti-GBM antibody disease, and mesangioproliterative glomerular nephritis, and animals models such as passive Heymann nephritis, puromycin aminonucleoside-induced proteinuria.

As used herein, the phrase “cytochrome P450” (CYP) is intended to include any member/isoenzyme of the CYP superfamily, which includes the mammalian CYP1, CYP2, CYP3 and CYP4 families (Omiecinski, et al., 1999, Toxicological Sciences 48:151-156). Thus, CYP includes the mammalian cytochrome P450 genes and their gene products, such as, P450 1A, 1A2, 1B1, 2B1, 2B2, 2B4, 2B5, 2B6, 2B11, 2A6, 2C6, 2C8, 2C9, 2C11, 2C18, 2C19, 2D6, 2E1, 3A4, 3A5, 3A7, 4A1 and 4B1. Of particular interest is the CYP isozyme CYP2B1 which is in glomeruli. A complete mRNA sequence and translation sequence of a human CYP2B1, GenBank accession number M29874, is provided herein as SEQ.ID.NO.:1. The phrase “cytochrome P450” (CYP) also includes allelic variants, site-specific mutants, and chimeric constructs of members of the CYP superfamily (Chang et al., 1997, Pharmacogenetics 7:211-221; Szklarz et al., 1995, Biochemistry 34:14312-14322; He et al, 1997, Biochemistry 36:8831-8839).

There are numerous CYP inhibitors/agents useful in the present invention for inhibiting a CYP superfamily member, all of which are included in the present invention.

As used herein the phrase “CYP inhibitor” includes any agent that inhibits at least one function of a CYP superfamily member. According to the present invention, CYP inhibitors include agents that directly interact with a CYP, indirectly interact with a CYP, directly or indirectly reduce the expression of a CYP, function in a biochemical pathway upstream of a CYP, and agents that function in a biochemical pathway downstream of a CYP. The interaction between a CYP and the CYP inhibitor may be a direct or an indirect interaction. The inhibitors useful in the present invention may function by interacting with the heme iron of CYP, thereby blocking or reducing the availability of the iron to serve as a catalytic iron source. The inhibitor agents of the present invention may inhibit CYP at the level of gene expression, gene transcription, translation, or CYP function.

Non-limiting examples of agents suitable for use in the present invention as CYP inhibitors include, cimetidine (CM), piperonyl butoxide (PB), safrole, isosafrole, myristicinfurfylline, diethyldithiocarbamate, chlormethiazol, piperine (PIP), disulfiram, diallyl sulfide, malotilate, allylmercaptan, methylprazole, orphenadrine, arylacetylenes, clorgyline and diphenhydramine. Generally the CYP inhibitors useful in the present invention are selected from the group consisting of cimetidine, piperonyl butoxide, piperine, diethyldithiocarbamate, chlormethiazol, disulfiram and diallyl sulfide. Preferably, the CYP inhibitor used in the present invention is selected from the group consisting of cimetidine, piperonyl butoxide, piperine, diethyldithiocarbamate, and chlormethiazol.

Without being limited by theory, it is possible that an increase in the generation of hydrogen peroxide results in direct oxidative attack on the heme moiety of CYP promoting the heme destruction and the release of iron. One mechanism by which inhibitors of the present invention may function is to inhibit this release of iron.

Additional inhibitors suitable for use in the compositions and methods of the invention include agents wherein the agent is a DNA, cDNA, RNA or polypeptide sequence. Suitable examples of such agents include: an antisense CYP sequence which inhibits transcription or translation of a CYP; transcription factors which decrease expression of a CYP gene; factors which affect translation of a CYP mRNA; factors which decrease the stability/half-life of a CYP mRNA molecule; factors which decrease the stability/half-life of a CYP polypeptide; and factors which interact with a CYP polypeptide, such as a polypeptide encoding an antibody which specifically interacts with an epitope of a CYP. The material and methods for producing these types of CYP inhibitors (DNA, cDNA, RNA and polypeptide) are known in the art and are included in the present invention.

For example, expression vectors comprising a sequence inhibitory to transcription of a CYP gene or comprising a sequence inhibitory to translation of a CYP mRNA are within the scope of the CYP inhibitors defined herein. Expression vectors suitable for the present invention may comprise an antisense CYP sequence, or a sequence encoding a negative regulator of transcription of a CYP gene.

Preferably the vectors of the invention comprise a CYP antisense sequence which will inhibit the mRNA of a CYP gene. Generally preferred targets for antisense technology are the CYP2 family members, more preferred are the CYP2B isozymes, and even more preferred is CYP2B1.

Vectors comprising a DNA sequence which encodes a polypeptide of a CYP dominant negative mutant, or a vector comprising a DNA sequence encoding a CYP mutant may also be useful inhibitors in the present invention.

Suitable vectors are known in the art and include, for example, mammalian expression vectors and viral vectors. Examples of viral vectors suitable for use in the present invention include: retroviruses; adenoviruses; adenoviral/retroviral chimeras; adeno-associated viruses; herpes simplex virus I or II; parvovirus; and reticuloendotheliosis virus. Other possible viral vectors may be derived from poliovirus, papillomavirus, vaccinia virus, lentivirus, as well as chimeric vectors incorporation favorable aspects of any two or more of the above viruses.

For guidance in the construction of gene therapy vectors and in the introduction thereof into affected individuals for therapeutic purposes see, for example, WO 99/05299, U.S. Pat. Nos. 5,631,236; 5,688,773; 5,691,177; 5,670,488; 5,601,818; and WO 95/06486.

Generally, methods are known in the art for viral infection of the cells of interest. The virus can be injected into a patient bearing a neoplasm, either at, into, or near the site of neoplastic growth. Preferentially, the treatment will be by direct intraneoplastic inoculation.

The compositions of the present invention further comprise a pharmaceutically acceptable carrier/vehicle. Pharmaceutically acceptable carriers/vehicles are known in the art and include aqueous solutions, non-toxic excipients, including salts, preservatives, buffers and the like, propylene glycol, polyethylene glycol, vegetable oil, injectable organic esters such as ethyloleate, water, saline solutions, parenteral vehicles such as sodium chloride and Ringer's dextrose, glycerol, lipids, alcohols.

Compositions of the present invention may be in any form known in the art, such as an orally digestible form, a sterile injectable form, forms suitable for delayed release, and forms that are enterically coated. Compositions of the invention may be in solid forms, including, for example, powders, tablets, pills, granules, capsules, sachets and suppositories, or may be in liquid forms including solutions, suspensions, gels and emulsions.

The compositions and methods of the present invention may be administered to a recipient/patient as a single dose unit, or may be administered in several dose units, for a period ranging from one day to several years. The dose schedule is dependent upon at least the severity of the glomerular disorder, as well as the mode of administration. The effective dose of the compositions of the present invention is further dependent upon the body weight (BW) of the recipient/patient and also upon the chosen inhibitor and are easily determined by one of skill in the art. Generally the compositions of the present invention are administered orally or intravenously.

The effective dose is that dose which decreases the symptoms of the glomerular disorder. Preferably, the treatment decreases proteinuria (the urinary excretion of protein). Generally, the effective dose reduces the proteinuria level to a value of less than about 500 mg /24 hour time period, preferably less than about 400 mg/24 hr time period, more preferably less than about 300 mg/24 hour time period, even more preferably less than about 200 mg/24 hour time period, still more preferably less than about 150 mg/24 hour time period. Methods for determining proteinuria are known in the art.

Generally for a glomerular disease, such as minimal change disease, treatment takes place during a time period ranging from about one day to about four years, preferably about one week to about three years, and even more preferably from about two weeks to about two years.

Generally for chronic proteinuric diseases, treatment takes place during a time period ranging from about one day to about twenty years, preferably from about one week to about fifteen years, and even more preferably from about two weeks to about ten years.

Generally, cimetidine is administered via an oral or intravenous route at a concentration in the range of about 10 to about 50 mg per kilogram of body weight, preferably about 15 to about 45 mg per kilogram of body weight, and more preferably about 20 to about 40. Generally cimetidine is given every six hours in a twenty four hour period.

Generally, piperine is administered by an oral or intravenous route in an amount ranging from about 100 to about 300 mg, preferably from about 125 to about 250 mg, and more preferably from about 150 to about 200 mg. Generally piperine is administered orally once every twenty-four hours.

The inhibitor DEDC is generally administered orally or intravenously at a concentration ranging from about 0.25 to about 2.5 g/m², preferably from about 0.4 to about 2.0 g/m², even more preferably from about 0.5 to about 1.8 g/m², and still more preferably from about 0.6 to about 1.6 g/M². Generally, DEDC is given once a day.

The inhibitor chlormethiazole is generally administered orally in an amount ranging from about 0.5 to about 3.5 g, preferably from about 0.75 to about 3.0 grams, even more preferably from about 1.0 to about 2.75 grams, and still more preferably from about 1.2 to about 2.4 grams. Generally, chlormethiazole is given once every twenty-four hours.

The compositions and methods of the present invention are suitable for any individual afflicted with a glomerular disorder. Suitable individuals include mammals such as, humans, dogs, cats, horses, cows, sheep, goats, pigs, rats and mice. The compositions and methods of the present invention are also suitable for use in any tissue or cell line that serves as a model for glomerular disorder, including glomeruli epithelial, endothelial and meager cells. Thus the present invention is useful to medical and health care professionals including, medical doctors, and veterinarians, as well as research scientists.

Administration of the present invention to a recipient may be by any method known in the art. Thus, administration of the present invention to a recipient may be by a route selected from oral, parenteral (including, subcutaneous, intradermal, intramuscular, and intravenous) and rectal. For increased efficacy, the compositions of the present invention may be administered via localized delivery to the kidney. Generally the present invention is administered to a recipient intravenously.

One method of the invention is directed to treating a patient having a glomerular disorder, such as a glomerular disease like minimal change disease. The method comprises the steps of administering a therapeutically effective amount of a composition to a patient, wherein the composition comprises an effective amount of an agent that inhibits a glomerular cytochrome P450, such as a cytochrome P450 2B family member. Preferably the agent is selected from the group consisting of cimetidine, piperonyl butoxide, piperine, diethyldithiocarbamate, chlormethiazole, and any combination thereof.

Another method in the invention is directed to treating a patient having a glomerular disorder, such as a glomerular disease. The method comprises the steps of administering a therapeutically effective amount of a composition to the patient, wherein said composition comprises an effective amount of an expression vector comprising a sequence encoding an inhibitor of a cytochrome P450, such as an antisense cytochrome P450 sequence. Preferably the antisense cytochrome P450 sequence is a cytochrome P450 2B family member, such as SEQ.ID.NO.:1.

Even another method of the invention is directed to treating a patient by administering a therapeutically effective amount of a composition to the patient, wherein the composition comprises an agent that inhibits a cytochrome P450 superfamily member and wherein said patient has a urinary protein excretion value of greater than about 300 mg in a twenty four hour time period. Preferably, the agent is selected from the group consisting of cimetidine, piperonyl butoxide, piperine, diethyldithiocarbamate, chlormethiazole, and any combination thereof. The cytochrome P450 superfamily member may be any cytochrome P450 of the 2B family. Treatment does not inhibit kidney function thus kidney function is preserved. Kidney function can be measured by BUN, plasma creatinine, glomerular filtration rate, or any combination thereof.

Still another method of the invention is directed to treating a patient by administering a therapeutically effective amount of a composition to the patient, wherein the composition comprises an expression vector comprising a sequence encoding a cytochrome P450 inhibitor, and wherein said patient has a urinary protein excretion value of greater than about 300 mg per twenty-four hour time period. Generally, the sequence is an antisense sequence of a cytochrome P450 2B family member, preferably the antisense sequence is a partial sequence of a cytochrome 2B family member.

Yet another method of the invention is directed to treating a patient by administering a therapeutically effective amount of a composition to the patient, wherein the composition comprises an agent that inhibits a cytochrome P450 superfamily member, and wherein said agent preserves kidney function as measured by BUN, plasma creatinine level, glomerular filtration rate, or any combination thereof.

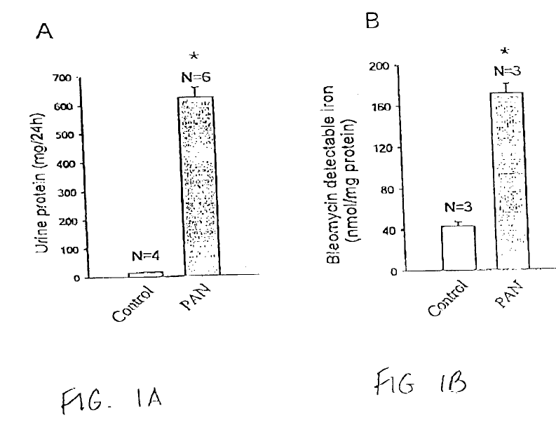

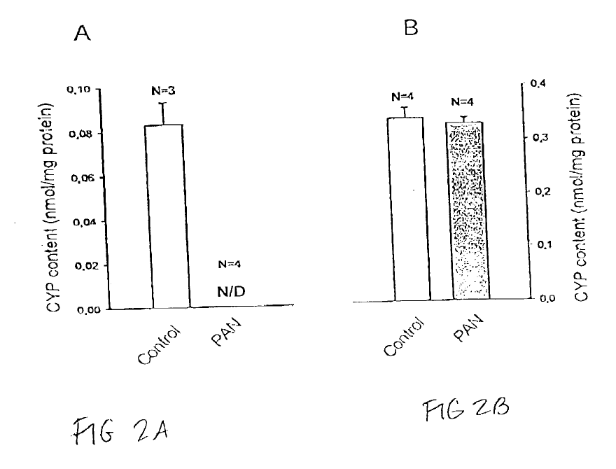

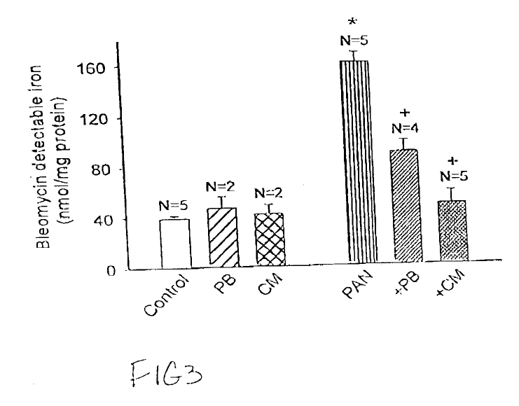

Referring now to FIGS. 1 and 2, a single injection of an effective dose of PAN to an animal, such as, a rat, induces a glomerular disorder referred to as PAN-induced nephrotic syndrome (NS). PAN-induced nephrotic syndrome in animals mimics MCNS in humans, and thus animals afflicted with PAN-induced NS serve as model systems for MCNS in humans. The effective amount of PAN is dependent on the body weight (BW) of the animal and generally is administered at a concentration of 7.5 mg/100 g BW. A single intravenous injection of PAN to rats results in marked proteinuria and renal morphological changes similar to minimal change disease in humans.

Intravenous administration of PAN results in nephrotic range proteinuria by the seventh day following administration, as shown in FIG. 1A. The catalytic iron content, as measured by the bleomycin detectable iron assay, is significantly elevated from a control value of 39 to 160 nmol/mg protein (N=3, P<0.01) in the glomeruli obtained from rats injected with PAN, as shown in FIG. 1B. In the liver, there is no difference in the level of CYP between the untreated and the PAN treated animals (FIG. 2B). There is a difference in the kidney CYP content of untreated and PAN treated animals however, wherein the CYP content of PAN treated rats was not detectable (FIG. 2A). Thus, administration of PAN results in a significant increase in the catalytic iron accompanied, at least, by a loss of CYP content in the glomeruli. This loss of CYP content is specific to the glomeruli as there is no difference detected in the levels of CYP content in the liver between PAN and control group of animals.

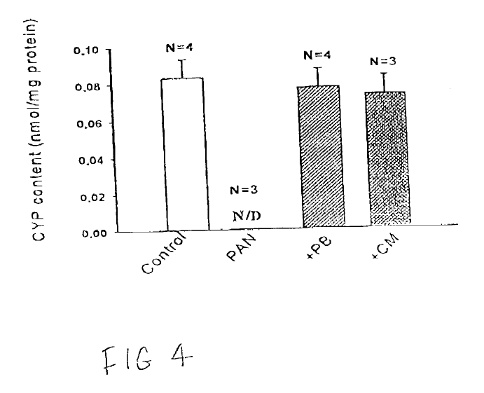

Referring now to FIGS. 3 and 4, the effects of two CYP inhibitors of the invention, cimetidine (CM) and piperonyl butoxide (PB), on the bleomycin detectable iron content in the glomeruli in PAN treated rats are shown. Without being limited by theory, it is believed that CM and PB function as inhibitors of CYP by interacting with the heme moiety of CYP. As shown in FIG. 3, both CM and PB significantly prevent the increase of bleomycin detectable iron in the glomeruli of PAN-treated rats. CM and PB also significantly preserve the loss of CYP content in the glomeruli of PAN-treated rats, as shown in FIG. 4. As discussed previously, CYP inhibitors in addition to CM and PB, are useful in the invention. These additional inhibitors include piperine, DEDC, and chlormethiazole.

Referring now to FIG. 5, the protective effect of two CYP inhibitors of the invention, CM and PB, on PAN-induced proteinuria is shown. Administration of PAN results in significant proteinuria on the fourth day with marked increases thereafter. The CYP inhibitor cimetidine is administered intraperitoneally one hour prior to PAN injection, and twice a day thereafter. The CYP inhibitor, PB, is administered intraperitoneally four hours prior to PAN injection, and every other day thereafter. As shown in FIG. 5, preincubation of the cells with the CYP inhibitor CM or the CYP inhibitor PB provides substantial protection against PAN-induced proteinuria.

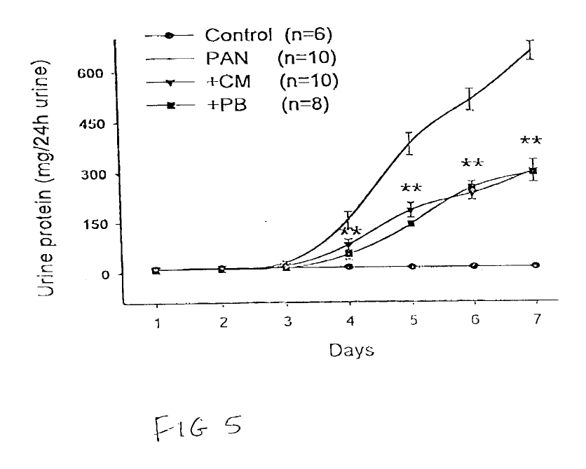

Referring now to Table 1 and FIGS. 6-8, the effect of two CYP inhibitors, CM and PB, on serum albumin, and renal function is shown. PAN treated rats exhibit significant weight gain at the time of sacrifice compared to control animals and those treated with the CYP inhibitors CM and PB (Table 1).

TABLE 1
Weight loss/gain of rats before and after treatments.

| | Before Treatment | After Treatment (day7) |
|---|---|---|
| | (g) | (g) |
| Control (N = 4) | 220 ± 6 | 265 ± 6 |
| PB (N = 4) | 223 ± 3 | 253 ± 4 |
| CM (N = 4) | 224 ± 5 | 253 ± 9 |
| PAN (N = 6) | 224 ± 1 | 291 ± 8* |
| PAN + PB (N = 6) | 221 ± 2 | 263 ± 4* |
| PAN ÷ CM (N = 6) | 228 ± 4 | 253 ± 9* |

Values are Means ± SE, *p < 0.05 compared with respective controls, + p < 0.05 compared with PAN.

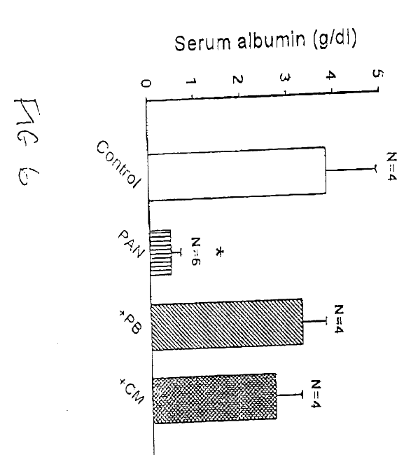

The serum albumin of PAN treated animals is markedly decreased, and this decrease is prevented by CYP inhibitors, such as CM and PB (FIG. 6). Renal function as measured by serum creatinine is similar in all the groups, while the BUN value is significantly elevated in the rats treated with both PAN and CYP inhibitors, compared to that of the control animals (FIG. 7).

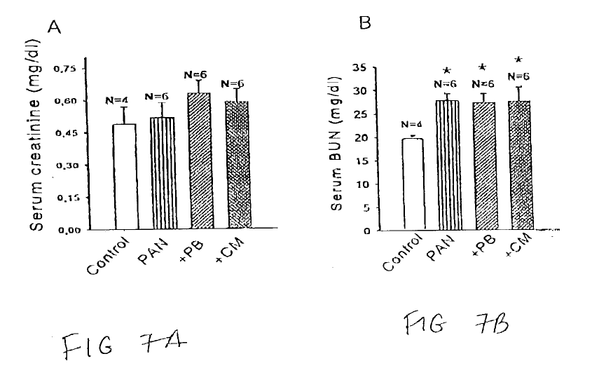

Referring now to FIG. 8, a role of CYP in an in vitro model of PAN induced cytotoxicity is shown. GEC cells are at least one of the specific cell-types injured in MCNS-induced nephrotoxicty. Exposure of GEC cells to a dose of 1.5 mM PAN results in a significant increase in the bleomycin detectable iron content. Treatment with either 2 mM CM or 25 uM PB drastically prevents the increase in the bleomycin detectable iron content. As a control, treatment with 1 mM RN, which has a similar structure as CM but is a weak inhibitor of CYP, is used to indicate this particular weak inhibitor of CYP does not prevent the marked increase in the bleomycin detectable iron content, and is not suitable for use alone. As weak CYP inhibitors may exert a combinatory effect with respect to CYP inhibition, it is within the scope of the invention to combine the use of more than one weak CYP inhibitor in the compositions and methods described herein to result in a less weak CYP inhibitor.

Referring now to FIG. 9, a potential role of hydroxyl radicals in PAN induced cytotoxicity in GEC is shown. Iron has been shown to participate in the generation of powerful oxidant species, such as the hydroxyl radical via the metal catalyzed Haber-Weiss reaction. As shown in FIG. 9, exposure of GEC to PAN leads to a significant increase in hydroxyl radical formation. The CYP inhibitors CM and PB markedly reduce the PAN induced hydroxyl radical formation in GEC. The weak CYP inhibitor RN does not reduce the PAN induced hydroxy radical formation.

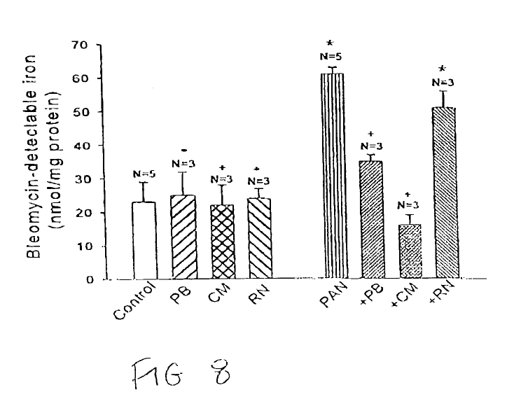

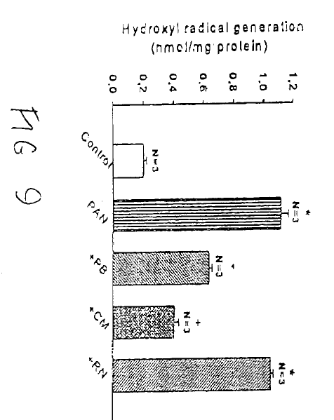

Referring now to FIGS. 10 and 11, PAN is cytotoxic to GEC in a manner that is both time and a dose dependent, as measured by the trypan blue exclusion assay (FIG. 10). However, two CYP inhibitors of the present invention, CM and PB, significantly reduce PAN induced cytotoxicity to the GEC (FIG. 11). RN, which has three times the H₂ receptor blocking activity as CM but is a weak inhibitor of CYP, does not exhibit protection against PAN-induced proteinuria.

The present inventors have discovered evidence of a close relationship between catalytic iron formation and the content of microsomal CYP, which is mainly involved in drug metabolism (Glassock et al, 1991, In the Kidney, 4^{th} Edition, B. M Brenner and F. C. Rector Editors, 1182-1279.). The CYP inhibitors of the present invention are markedly effective in reducing PAN-induced proteinuria in rats, and also in PAN-induced cytotoxicity to GEC. Thus, the present inventors propose that microsomal CYP is a major source of catalytic iron.

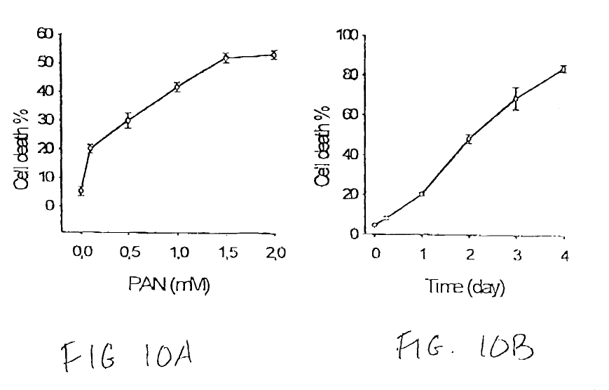

Referring now to FIGS. 12A and 12B immunohistochemistry and western blot data, respectively, of the identification and localization of CYP2B1 in the rat kidney are provided. These figures reveal that CYP2B1 is present in the glomeruli but not in the tubules of the kidney. The inventors note this is the first time CYP2B1 has been documented/reported to be in rat glomeruli. Previous studies on CYP isoforms by others indicate a presence of CYP localized only in the proximal tubules but not in the glomeruli.

Shown in FIG. 13A, are the effects of CYP450 inhibitors on the proteinuria (mg/24h) in rats treated with PAN. Symbols are: ●=control, N=6; [symbol] =PAN, N=10; ▾=CM, N=6; ▪=PIP, N=8. Treatment with the CYP2B1 inhibitors PIP and C<provide significant protection against PAN-induced proteinuria on day 4 and throughout the course of the study. FIG. 13B shows the effects of CYP inhibitors on the serum albumin (g/dL) in PAN-treated rats. Values are means±SE, N=6 to 10. *=P<0.05 compared with the rats treated with PAN alone. +=P<0.05 compared with control rats. As seen in FIG. 13B, injection of PAN results in a significant decrease in serum albumin level and this decrease was prevented by the administration of CYP inhibitors.

PAN treated animals developed ascites and significant weight gain at the time of sacrifice compared to the control animals and those treated with the CYP inhibitors PIP and CM.

Renal function as measured by serum creatinine was similar in all the groups (control, 0.72±0.2; PAN, 0.85±0.2; +PIP, 1.10±0.6; +CM, 0.69±0.1 mg/dL), while the BUN was significantly elevated in rats treated with PAN and CYP inhibitors in comparison to control animals (control, 21±3; PAN, 33±4; +PIP, 38±3; +CM, 28±3 mg/dL; P<0.05 vs control) (data not shown).

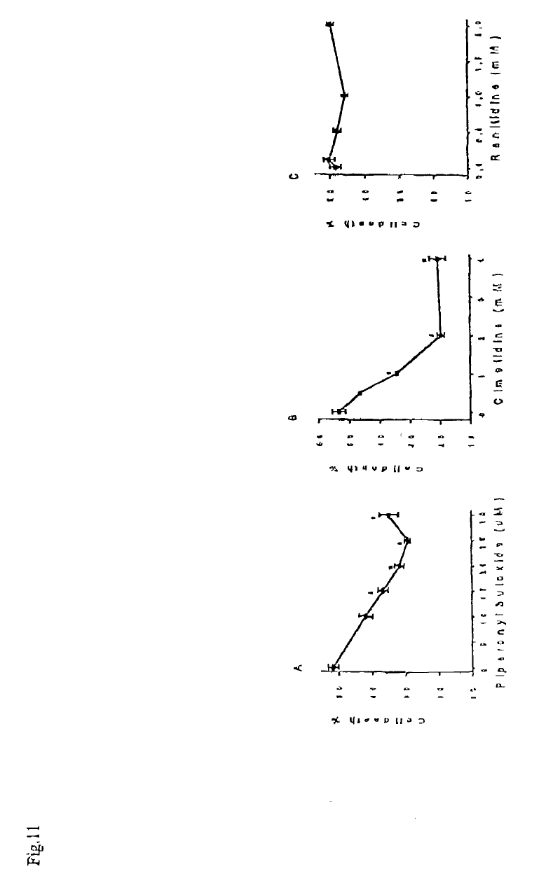

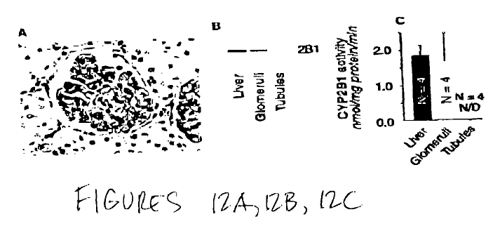

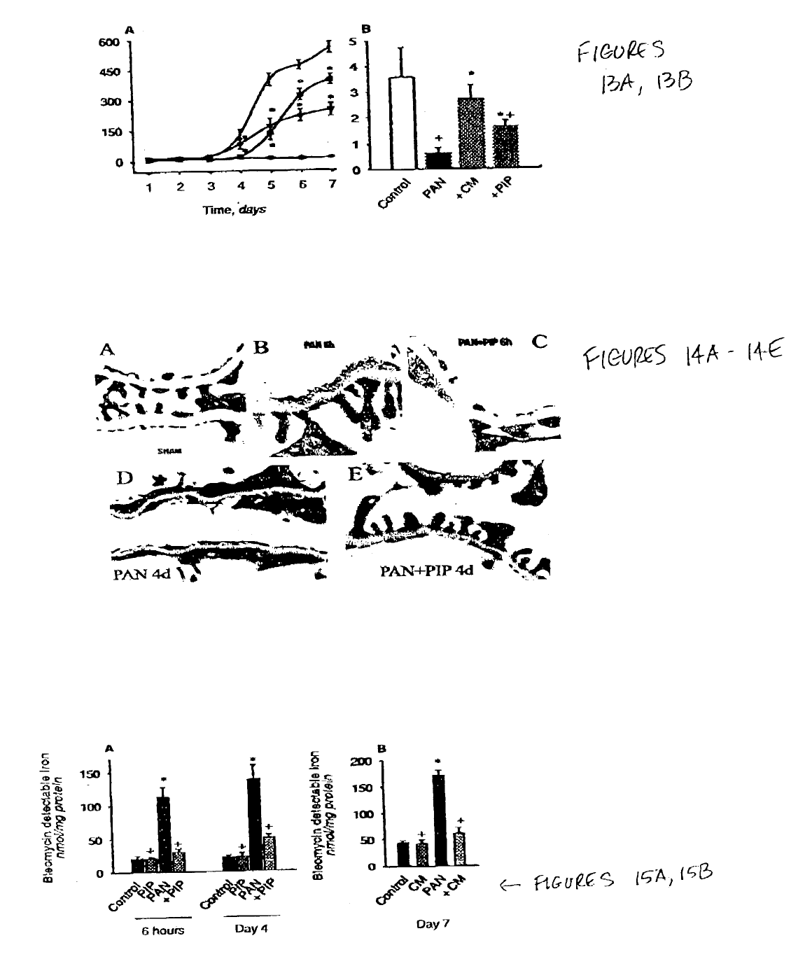

Though not intending or wishing to be limited or bound by theory, the present inventors postulate that the generation of H₂O₂ from the interaction of CYP with PAN results in inactivation of the CYP with a subsequent release of iron and heme. Thus, the in situ generation and localization of reactive oxygen metabolites (ROM) in the glomeruli of rats injected with PAN was investigated. By use of an in vivo ultrastructural cerium histochemistry technique for H₂O₂ localization, the present inventors found a presence of electron-dense granular precipitate (a H₂O₂-cerium complex) diffusely distributed throughout the glomerular basement membrane at one hour (data not shown) and more extensively at 6 hours following PAN injection (FIG. 14B). The cerium-H₂O₂ reaction product in the glomerular basement membrane was significantly attenuated in the PAN rats treated with the CYP2B1 inhibitor PIP (FIG. 14C) while control animals did not show any reaction product in their basement membranes (FIG. 14A). Shown in FIG. 14D, on the fourth day with the onset of proteinuria there was the marked fusion of the foot processes noted in the PAN treated animals. Of interest is that treatment with PIP resulted in significant preservation of the foot processes (FIG. 14E). These results provide direct evidence that the interaction between CYP2B1 and PAN leads to the generation of H₂O₂ in the glomeruli, especially in the glomerular basement membrane. The precise location of the production of H₂O₂ is not demonstrable by this technique as H₂O₂ is well known to diffuse rapidly across the cell membrane.

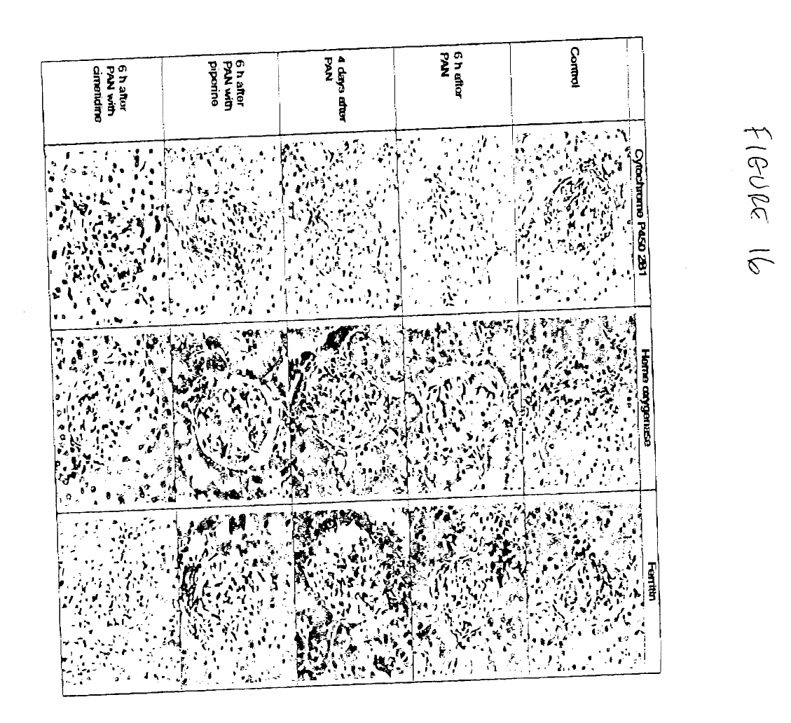

The effect of the CYP inhibitors PIP and CM on the bleomycin-detectable iron content in the glomeruli of PAN treated rats was then examined. FIGS. 15A and 15B show the effect of CYP inhibitors on the bleomycin detectable iron content in the glomeruli of PAN-treated rats at 6 hours, on day 4, and on day 7. Values are means±SE, N=3 to 5, * P<0.05 compared with control rats, +=P<0.05 compared with rats treated with PAN alone. As illustrated, the bleomycin-detectable iron content in the glomeruli of PAN treated animals is markedly elevated in comparison to control animals. Also clearly illustrated in FIGS. 15A and 15B, treating the animals with either PIP or CM significantly prevented this increase in bleomycin-detectable iron content in the glomeruli. Taken together with FIGS. 14A-14E, the present date strongly suggest CYP2B1 plays an important role in PAN-induced nephrotic syndrome by serving as a major site for the generation of ROM and a significant source of iron capable of catalyzing free radical reactions.

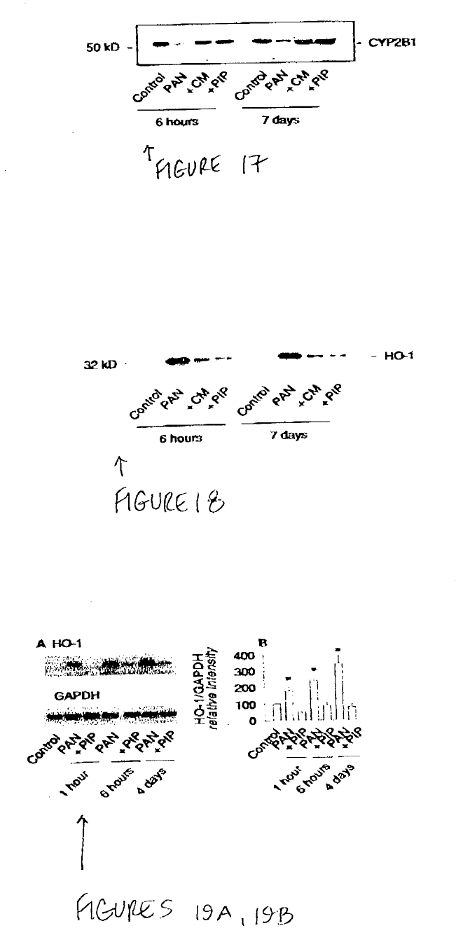

Referring now to FIG. 16 (left column) and FIG. 17, the CYP inhibitors which markedly decreased the H₂O₂ generation and prevented the increase in bleomycin detectable iron (FIGS. 14 and 15) also prevented the loss of CYP2B1 content in the glomeruli (FIGS. 16 and 17). FIG. 16 provides the immunohistochemistry data for CYP2B1 (left column), heme oxidase (HO-1) (middle column), and ferritin (right column) following PAN treatment +/−piperine, +/−cimetidine, at 6 hours and at day 4. FIG. 17 is a western blot analysis of CYP2B1 protein in glomeruli of PAN treated rats with or without administration of CM and PIP at 6 hours and 7 days after PAN injection.

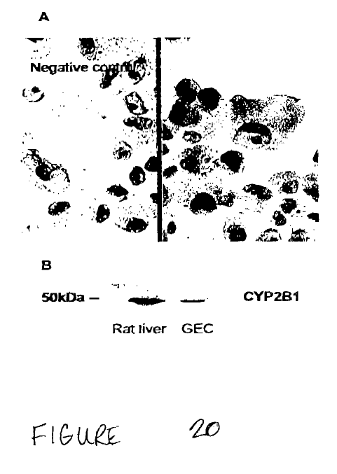

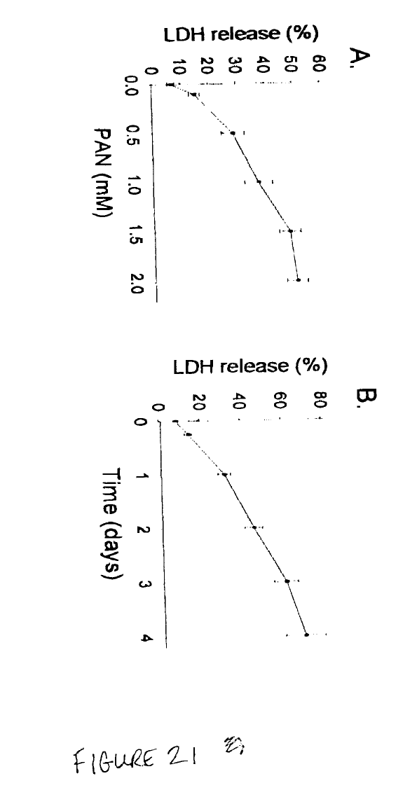

Oxidant stress induces heme oxidase (HO-1), the rate-limiting enzyme in heme degradation, which has been shown to be protective in several nephrotoxic models fo renal injury. Referring now to the FIG. 16 (middle column), FIG. 18, and FIG. 19, administration of PAN resulted in a marked induction of HO-1 in the glomeruli and tubules. FIG. 18 is a western blot analysis of heme oxidase (HO-1) protein in the glomeruli of rats injected with PAN +/−administration of CM and PIP. The blot is representative of results from 6 to 10 animals per experimental group, 6 hours after and 7 days after PAN injection. FIGS. 19A and 19B show the induction of HO-1 mRNA in the kidney of PAN-treated rats and the effect of CYP2B1 inhibitor PIP. FIG. 19A shows the products following RT-PCR of Ho-1 mRNA (top portion of figure) and GAPDH mRNA (bottom portion of figure) run on a 2% agarose gel. FIG. 19B provides the OD values for HO-1 mRNA at each time point depicted and are expressed compared with the corresponding GAPDH (control) values. The OD values are means±SE, N=3, P<0.05 compared with control level. As can be seen, HO-1 mRNA expression in the kidney was significantly increase at one hour after PAN injection and continued to increase until day 4. Administration of the CYP2B1 inhibitor PIP markedly reduced the HO-1 mRNA induction.

The degradation of CYP hemeprotein results in the release of iron, which stimulates ferritin synthesis. Ferritin provides a storage site for iron and sequesters circulating free iron, thus preventing it from participating in subsequent oxidant injury. The present inventors have found in PAN treated rats there is a marked decrease in CYP2B1 content in the glomeruli and significant increase in the content of ferritin both in the glomeruli and the tubules compared to the control animals, as demonstrated by immunohistochemistry (FIG. 16, right column). As shown, the CYP inhibitors PIP and CM, which preserved CYP2B1 content in the glomeruli, markedly reduced the up-regulation of ferritin.

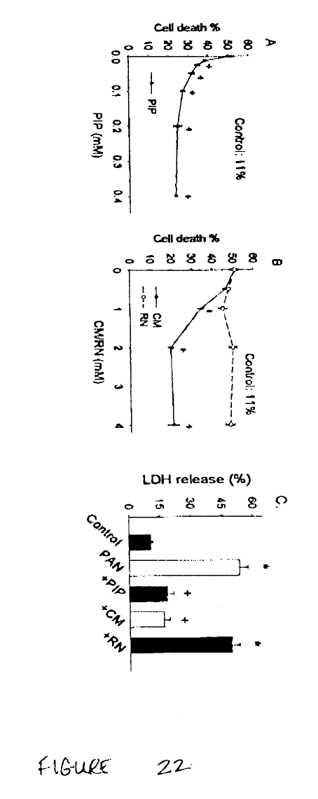

In keeping with the present inventors' in vivo studies which unexpectedly identified a presence of CYP2B1 in rat glomeruli (FIG. 12B), CYP2B1 was detected in vitro by immunohistochemistry and western blot analysis in GEC (FIGS. 20A and 20B, respectively).

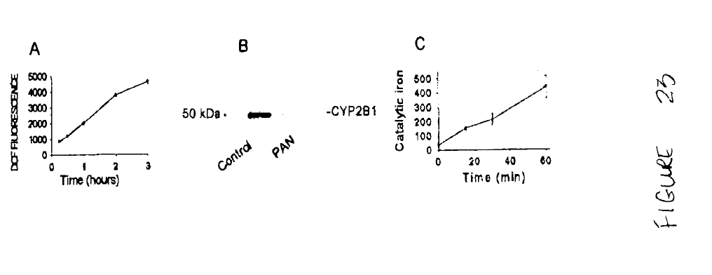

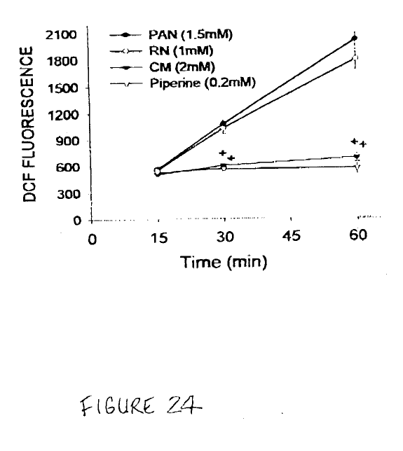

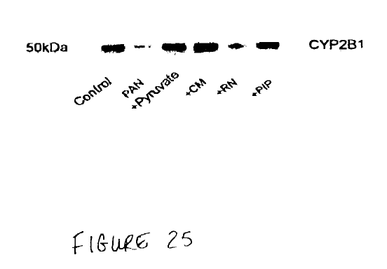

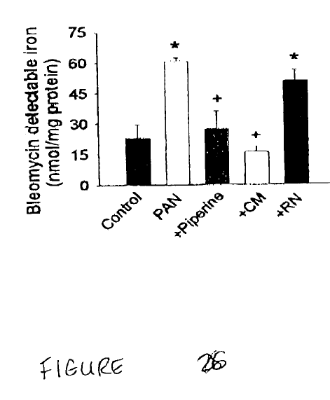

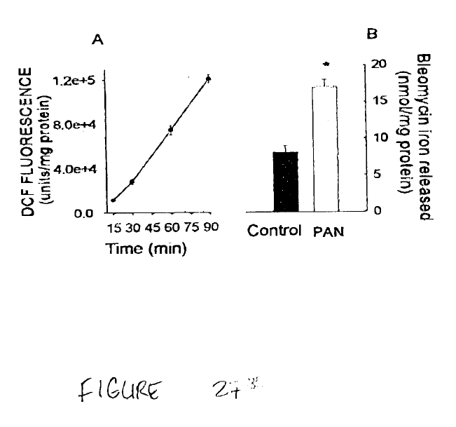

Exposure of GEC to PAN resulted in cell death in a time and dose dependent manner as measured by LDH release (FIGS. 21A and 21B) and trypan blue exclusion assay (FIG. 10B). Shown in FIGS. 22A, 22B and 22C, preincubation of CYP2B1 inhibitors PIP and CM markedly reduced the PAN-induced cytotoxicity. RN, which has three times the H₂-receptor blocking activity as CM but is a weak CYP inhibitor, did not show any protection. Incubation of GEC with PAN (1.5 mM) resulted in a marked increase in H₂O₂ generation in a time dependent fashion (FIG. 23A) and a significant reduction of CTP2B1 content after 2 hours of incubation (FIG. 23B) associated with a significant increase in catalytic iron (FIG. 23C). FIG. 23A is time course of H₂O₂ generation in GEC exposed to 1.5 mM PAN (the difference in the mean DCF fluorescence between treated and untreated cells was calculated as fluorescence increase due to PAN induced H₂O₂ generation); FIG. 23B is a western blot analysis of CYP2B1 content in control GEC and PAN-treated GEC (1.5 mM for 2 hr at 37° C.); and FIG. 22C is a time course of catalytic iron release (nmol/mg protein) from GEC exposed to PAN. The CYP inhibitors PIP and CM, but not RN (used as a control for CM), significantly decreased the intracellular H₂O₂ generation (FIG. 24), preserved the CYP2B1 content (FIG. 25) and prevented the increase in catalytic iron (FIG. 26).

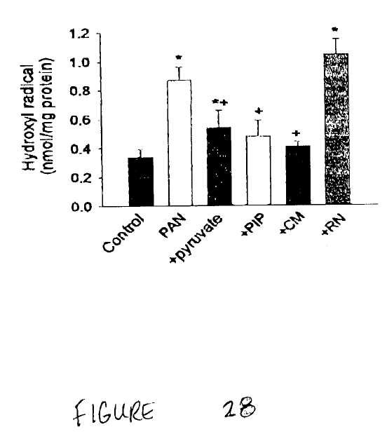

The generation of H₂O₂ and catalytic iron from microsomal CYP isolated from GEC was also analyzed. Treatment of the microsomal fraction with PAN 91.5 mM) results in H₂O₂ generation in a time dependent fashion associated with a marked increase in catalytic iron release (FIG. 27A, 27B).

As with the in vivo studies discussed above, the present inventors proposed that if CYP were indeed: 1) a site for the generation of H₂O_{2,}; and 2) a source of iron, then pre-incubation with a H₂O₂ scavenger such as pyruvate should result in preservation of the CYP2B1 content with a marked decrease in catalytic iron. The inventors discovered that indeed, administration of pyruvate (10 mM) was found to markedly reduce the H₂O₂ generation in GEC preserve the CYP2B1 content, significantly decrease the catalytic iron, and prevent PAN-induced cytotoxicity (data not shown).

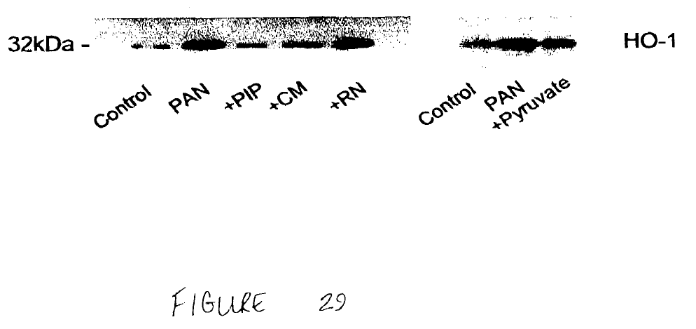

Catalytic iron is critical in the generation of powerful oxidant species such as the hydroxyl radical via the metal catalyzed Haber-Weiss reaction. Therefore, the present inventors analyzed the generation of hydroxyl radical in GEC exposed to PAN. As shown in FIG. 28, incubating GEC with PAN (1.5 mM) resulted in significant production of hydroxyl radical at 1 hour. Also shown in FIG. 28, the CYP2B1 inhibitors PIP and CM, but not RN, significantly reduced the hydroxyl radical formation in GEC exposed to PAN, as did the H₂O₂ scavenger pyruvate. Values are means±SE, N=3, *=P<0.05 compared to control, +P<0.05 compared to GEC exposed to PAN alone.

As discussed in the description of FIGS. 16, (middle column) 18 and 19, HO-1 is a rate-limiting enzyme that has been shown to play a beneficial role in tissue injury. FIG. 29 provides the results from a western blot analysis of HO-1 induction in GEC exposed to PAN +/−the CYP inhibitors PIP (0.2 mM), CM (2 mM) and the H₂O₂ scavenger pyruvate (10 mM). The blot shown is representative of the results from 3 groups of studies. As can be seen in FIG. 29, GEC exposed to PAN showed marked induction of HO-1. CYP inhibitors (PIP and CM but not RN) and H₂O₂ scavenger (pyruvate) significantly prevented the induction of HO-1 induced by PAN.

The present inventors have thus provided evidence for a role of CYP2B1 in PAN induced cytotoxicity by serving as a site for the generation of ROM and a significant source of catalytic iron. Without being bound or limited by theory, the inventors believe the interaction of PAN with the microsomal CYP2B1 results in the generation of H₂O₂ which causes destruction of the CYP2B1 with the subsequent release of free iron and heme. The inventors further propose that this release of iron catalyzes the formation of hydroxyl radicals and other powerful oxidants while the released heme leads to induction of HO-1.

It is not outside the scope of the present invention that other intracellular sources of iron participating in ROM mediated glomerular injury may exist. For instance, mitochondrial cytochromes, iron-sulfur protein and other iron containing proteins may also serve as alternative sources of iron.

All references cited herein, including research articles, all U.S. and foreign patents and patent applications, parent application Ser. No. 09/672,296, filed Mar. 29, 2000, entitled “Compositions and methods for treating glomerular disorders”, are specifically and entirely incorporated by reference.

### EXAMPLES

The following in vivo and in vitro Examples (also found in Liu et al., 2002, Kidney Intl., 62:868-876, and Liu et al., 2003, Exptl. Nephrology Nephron, in press, both of which are incorporated herein by reference) are provided merely to illustrate the present invention, and are not meant to limit the scope of the claims in any way.

### Example 1

### PAN-Induced Nephrotic Syndrome

Male Sprague-Dawley rats weighing 200-250 g were injected either saline or a single intravenous injection of PAN (Sigma, St. Louis) in a dose of 7.5 mg/100g BW (day 0) as described in Ueda et al, 19996, Kidney Intl. 49:370-373. Animals were housed in separate metabolic cages and allowed free access to rat chow (Purina). Daily urine protein excretion was determined and animals were sacrificed on day 7. Blood was obtained for the measurement of serum albumin and the evaluation of renal function as measured by blood urea nitrogen (BUN) and plasma creatinine. Cell fraction of the glomeruli was prepared for bleomycin detectable iron assay. The microsome fraction was utilized for the measurement of CYP content.

### Example 2

### Inhibition of cytochrome P450

Two different general CYP inhibitors were used, cimetidine (CM) and piperonyl butoxide (PB). Cimetidine has imidazole and cyano groups that inhibit CYP by interacting with the heme moiety. This effect of CM is specific for CYP, as it does not interact with other heme enzymes. To determine the effect of the CYP inhibitors in PAN- induced nephrotic syndrome, CM (120 mg/kg BW) was administered intraperitoneal 1 h prior to PAN injection and then twice a day. Piperonyl butoxide (PB) which yields a metabolite that binds to the heme moiety of CYP, was given intraperitoneal (400 mg/kg BW) 4h before PAN injection and then every other day.

Also, the CYP2B1 inhibitor piperine (PIP), a methylenedioxyphenyl compound found in black and red pepper was used. PIP is believed to inhibit CYP2B1 by forming an inhibitory complex therewith. PIP was administered at a dose of 85 mg/kg of body weight (BW) by an intraperitoneal injection 16 hours prior to PAN injection and then once a day.

As a control for CM, ranitidine (RN) which has three times the H₂-receptor blocking activity as CM, inhibits CYP either weakly or not at all.

### Example 3

### Isolation of Glomeruli

Glomeruli were isolated by a combination of sieving and differential centrifugation as described in Ueda et al, 19996, Kidney Intl. 49:370-373, and Baliga et al, 1992, Am. J. Physiol. 263:214-221. Glomeruli isolated from two rats were pooled together (as N of 1) for the isolation of microsomes.

### Example 4

### Bleomycin Detectable Iron Assay

Iron capable of catalyzing free radical reactions was measured by bleomycin detectable iron assay as described by Gutteridge et al, 1981, Biochem. J. 199:263-265, Gutteridge et al, 1982, Biochem. J. 206:605-609, Baliga et al, 1993, Bioche. J. 291:901-905, Baliga et al, 1996, Kidney Int. 49:362-369, Baliga et al, Kidney Int. 53:394-401, Ueda et al, 1996, Kidney Int. 49:370-373, Baliga et al, 1998, Kidney Int. 54:1562-1569.

### Example 5

### Scavenging of H₂O₂

Confluent GEC monolayers were washed twice with HBSS. The cells were then incubated with H₂O₂ scavenger pyruvate (10 mM) for 60 minutes at 37° C.

### Example 6

### Preparation of Microsome Fraction

Glomeruli isolated from rat kidneys were suspended in an extraction buffer containing 20 mM Tris-HCl, pH 7.4, 0.25 M sucrose, 1 mM EDTA and protease inhibitor cocktail (1 μl/25 mg protein) from Sigma, St. Louis, Mo.) and frozen at −80° C. Subsequently, the glomeruli were thawed and sonicated. The homogenate was centrifuged at 15,000×g for 20 min at 4° C. and the precipitate discarded. The microsomes were sedimented by centrifugation of the supernatant at 105,000×g for 60 min at 4° C. The sedimented microsomal pellet was resuspended in above extracting buffer to give a protein concentration of approximately 10 mg/ml.

CYP content was measured by the method of Omura and Sato, 1964, JBC 239:2370-2378. In brief, suspension of microsome from the glomeruli was diluted to about 1 mg of protein per ml with the assay buffer (0.1 M potassium phosphate buffer, pH 7.25, 20% glycerol, 0.2% tergitol). After recording the base-line, the sample was reduced with a few crystals of dithionite, and followed by CO bubbling for about 1 min. The CO difference spectrum of reduced microsomes was recorded on a Shimadzu UV-2101PC spectrophotometer. The peak absorbance at A450ₙₘ was measured, and the amount of CYP was determined using the extinction coefficient of 91 mM⁻¹cm⁻¹.

### Example 7

### Cytochrome P450 2B1 Activity

CYP2B1 activity was assayed by measuring resorufin formation from 7-pentoxyresorufin according to the procedure of Burke et al. (Biochem. Pharmacol. 1985, 34:3337-3345.). The reaction mixture containing the microsomal protein, 5 μmol/l 7-pentoxyresorufin and 0.1 mol/L phosphate buffer were equilibrated for one minute at 37° C. The reaction was started by the addition of 10 μl of 50 mmol/L NADPH. The accumulation of resorufin was measured by setting the fluorimeter excitation and emission wavelengths at 550 and 582 nm. The formation of resorufin was calculated by comparing the rate of increase in fluorescence of test samples to the fluorescence of known amounts of resorufin.

### Example 8

### Western Blot

The microsome fraction obtained was subjected to SDS-page electrophoresis in 1 mm slab gels. The separated forms were transferred from gel to a nitrocellulose sheet using a Mini Trans-blot electroblotting unit. The primary antibodies were monoclonal anti-HO-1 (OSA-111, StressGen, Victoria, BC, Canada) and monoclonal anti-CYP2B1 (PM25, Oxford Biomedical Research, Oxford, Mich., USA). Subsequent to primary and secondary antibody (peroxidase-labeled) treatment, the blots were visualized by enhanced chemiluminescence method.

### Example 9

### Immunochemistry of GEC

Cultured rat GEC were fixed in 95% alcohol for 5 minutes and attached on a glass slide by cytospin technique. The cells were dehydrated and permeabilized with 0.5% triton X-100 for 30 minutes. The immunolabelling and visualization was performed by ABC method.

### Example 10

### Immunochemistry of Kidney Cortical Sections

Kidney cortical sections were fixed in 10% buffered formalin for 6 hours and embedded in paraffin. After deparaffinization and antigen retrieval, the sections were immunolabeled and visualized according to an avidin-biotin complex (ABC) method. The primary antibody for detection of ferritin was polyclonal anti-Ferritin (L subunit) antibody (605022; Boehringer Mannheim Corp., Indianapolis, Ind., USA).

### Example 11

### Ultrastructural localization of H₂O₂

The principle of this method is that cerium ions combine with H₂O₂ generated by the cell that results in a precipitate with properties suitable for ultrastructural histochemistry. Localization of H₂O₂ generation was performed following the procedure of Neale et al (PNAS, USA, 1993, 90:3645-3649.). Following PAN treatment, the left kidney was perfused at 37° C. by retrograde aortic perfusion with 4 ml of each of the following solutions: a) PBS; b) 20 mmol Tris-maleate buffer (pH 7.2) containing 7% sucrose (TMS); c) TMS containing 1 mmol aminotriazole; d) TMS containing 10 mmol aminotriazole, 1 mmol cerium chloride heptahydrate and 2.56 mmol B-NADPH; and e) TMS followed by 10 mmol of 2.5% glutaraldehyde as the fixative. Pieces of the kidney were fixed further for 2 hours in glutaraldehyde and embedded in Epon. Sections from the tissue block were routinely stained with 4% aqueous uranyl acetate and alkaline lead, and examined under a Leo 906 electron microscope.

### Example 12

### Reverse transcription PCT (RT-PCR)

RNA was isolated from the kidney cortex using TRIzol Reagant (Gibco, BRL, Gaithersburg, Md., USA). Revers transcription of 0.5 mg of total RNA was carried out using Moloney murine leukemia virus reverse transcriptase (Gibco, BRL). The resulting first-strand cDNA preparations were used as templates for PCR. HO-1 primer described by Paschen et al (Neurosci Lett, 1994, 180:5-8.) were purchased from Gibco, BRL. The sense primer was: %′-TGGAAGAGGAGATAGAGCGA-3′, and the antisense 5′-TGATGAGCAGGAAGGCGGTC-3′. The amplification product was 451 bp. GAPDH sense was: 5′-TCCCTCAAGATTGTCAGCAA-3′; and GAPDH antisense: 5′-AGATCCACAACGGATACATT-3′. The PCR conditions were a hot start of 94° C. for 5 minutes, followed by cycling of 1 minute at 60° C., 1 minute at 72° C. and 1 minute at 92° C. for 30 cycles. The reaction was finished with 2 minutes at 60° C. and 10 minutes at 72° C. The PCR products were visualized on a 2% agarose gel using EtBr and UV transmission.

### Example 13

### BUN and Creatinine Measurements

BUN and creatinine were measured by using assay kits from Sigma.

### Example 14

### Measurement of Intracellular H₂O₂ Generation in GEC

The principle of this method is that the oxidation of 2′,7′-dichlorofluorescin-diacetate (DCFH-DA) in the presence of H₂O₂ produces the highly fluorescent compound 2′,7′-dichlorofluroescin (DCF) which can be measured by fluorometer. The intracellular generation of H₂O₂ in GEC was tested utilizing the microplate assay procedure described by Rosenkranz et al (J. Immunol. Methods, 1992, 156:39-45.). In brief, confluent GEC were harvested by trypsin and suspended in HBSS. After an equilibration period at 37° C. with constant shaking, they were transferred to a microplate (2×10⁵-1×10⁶ cells/well) and then incubated at 37° C. with equal volumes of DCFH-DA (10 μl/ml) and PAN (1.5 mM) for the planned time period. At the end of the incubation, the fluorescent intensity of the cell suspension was read using a fluorescence spectrophotometer (wavelength 485/535 nm) capable of reading microtiter plates.

### Example 15

### Measurement of Hydroxyl Radical Formation

2-deoxy-D-ribose in a final concentration of 3 mM was added to the medium just prior to the incubation. At the end of the incubation, the incubation medium was collected for the measurement of hydroxyl radical formation by deoxyribose degradation method as in Baliga et al (Kidney Intl. 1996, 50:1118-1124.).

### Example 16

### Cell Culture

Rat GEC (kindly provided by both Dr. Saulo Klahr, Washington University School of Medicine, St. Louis, Mo., and Dr. S. Kasinath, University of Texas Health Science Center, San Antonio, Tex.) were maintained in RPMI-1640 medium supplemented with 10% FBS, 15 mM HEPES, insulin, penicillin, streptomycin and L-glutamine in a humidified atmosphere of 95% air-5% CO₂ at 37° C. and fed at intervals of 3 days (Kasinath et al, 1995, Biochem. Biophys. 318(2):286-294). The cells were maintained in 75 Cm² tissue culture flasks and the monolayers were subcultured using 0.05% trypsin-0.53 mM EDTA in Hank's balanced salt solution (HBSS). For the experimental study, the cells were grown in 12-well tissue culture plate until confluence.

### Example 17

### PAN-Induced Cytotoxicity

On the day of experiment, the medium was discarded and the confluent GEC monolayer was washed twice with HBSS. The cells were then incubated with various concentrations of PAN (0, 0.1, 0.5, 1.0, 1.5, and 2.0 mM in HBSS) for different periods of time (0, 6 hours, 1 day, 2 days, 3 days and 4 days) at 37° C. At the end of the incubation, the incubation medium was discarded and the GEC monolayer was harvested by trypsinization with 0.05% trypsin-0.53 mM EDTA for 5 min at 37° C. Isolated cells were suspended in HBSS to give about 106 cells/ml. PAN-induced cytotoxicity to GEC was measured by the trypan blue exclusion assay (Baliga et al, 1996, Kidney Intl. 50:1118-1124) and lactate dehydrogenase (LDH) release assay (Baliga et al., 1998, Kidney Intl. 53:394-401.). The latter is the percentage of LDH released into the medium, to total LDH recovered from both medium and cellular fraction.

### Example 18

### Effect of CYP Inhibitors on the PAN Induced Cytotoxicity

Confluent GEC monolayers were washed twice with HBSS and then incubated with various concentrations of CM, ranitidine (RN, as a control of CM) for 30 min and PB for 60 min at 37° C. After the incubation, the cell monolayers were washed twice with HBSS and then incubated with cytotoxic dose of PAN in HBSS for a period of time necessary to induce consistent cytotoxicity (1.5 mM/ml, 48 h, based on the concentration and time course studies) at 37° C. PAN induced cytotoxicity on GEC was measured by trypan blue exclusion assay.

### Example 19

### Effect of CYP Inhibitors on PAN Induced Catalytic Iron Release

Confluent cell monolayer was washed three times with Chelex-treated HBSS to remove as much contaminating iron as possible. The GEC monolayer was then incubated in Chelex-treated HBSS with cytotoxic dose of PAN for a period time at 37° C. before substantial cell killing and after significant iron release occurs (1.5 mM, 60 min, based on a time course study on iron release induced by PAN, data not shown). The incubation medium was then collected for the measurement of catalytic iron using bleomycin detectable iron assay as mentioned above. To determine the effect of CYP inhibitors on the iron release, GEC monolayer was preincubated with CM (2 mM), RN (1 mM, as a control of CM) for 30 min or PB (25 uM) for 60 min in Chelex-treated HBSS at 37° C. After the incubation, the medium with CYP inhibitor was discarded and then the cell monolayers were washed twice with chelex-treated HBSS prior to the incubation of PAN.

### Example 20

### Effect of CYP Inhibitors on PAN Induced Hydroxyl Radical Formation

Confluent GEC monolayer was washed twice with HBSS and then incubated with 1.5 mM PAN in HBSS for a period of time before substantial cell killing occurs (1 h) at 37° C. 2-deoxy-D-ribose in a final concentration of 3 mM was added to the medium just prior to the incubation. At the end of the incubation, the incubation medium was collected for the measurement of hydroxyl radical formation by deoxyribose degradation method described in Baliga et al, 1996, Kidney Int. 50:1118-1124. To determine the effect of CYP inhibitors on the hydroxyl radical formation, GEC monolayers were preincubated with CM, RN or PB as mentioned above.

### Example 21

### Statistical Analysis

The values are expressed as mean±standard error (SE). Statistical analysis was performed using unpaired t-test (for only two groups) and analysis of variance (ANOVA; for more than two groups). Statistical significance was considered at P<0.05.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which this invention pertains. Thus, the specification and examples should be considered exemplary only with the true scope and spirit of the invention indicated by the following claims.

We claim:

1. A method for treating a patient, the method comprising the steps of:
(a) administering a therapeutically effective amount of a composition to a patient having a glomerular disorder, wherein the composition comprises an effective amount of an agent that inhibits a glomerular cytochrome P450.

2. The method of claim 1, wherein said cytochrome P450 is a CYP2B family member.

3. The method of claim 1, wherein said agent is selected from the group consisting of cimetidine, piperonyl butoxide, piperine, diethyldithiocarbamate, chlormethiazole, and any combination thereof.

4. The method of claim 1, wherein said glomerular disorder is a glomerular disease.

5. The method of claim 4 wherein said glomerular disease is minimal change disease.

6. The method of claim 1 wherein said administering is oral or parenteral.

7. A method for treating a patient, the method comprising the steps of:
(a) administering a therapeutically effective amount of a composition to a patient having a glomerular disorder, wherein said composition comprises an effective amount of an expression vector comprising a sequence encoding an inhibitor of a cytochrome P450.

8. The method of claim 7 wherein said sequence is an antisense cytochrome P450 sequence.

9. The method of claim 8 wherein said cytochrome P450 sequence is a cytochrome P450 2B family member.

10. The method of claim 9 wherein said sequence is SEQ.ID.NO.:1.

11. The method of claim 7, wherein said glomerular disorder is glomerular disease.

12. The method of claim 8, wherein said glomerular disease is minimal change disease.

13. The method of claim 7., wherein said administering is parenteral.

14. A method for treating a patient, the method comprising the steps of:
(a) administering a therapeutically effective amount of a composition to a patient, wherein said composition comprises an agent that inhibits a cytochrome P450 superfamily member, and wherein said patient has a urinary protein excretion value of greater than about 300 mg in a twenty four hour time period.

15. The method of claim 14, wherein said agent is selected from the group consisting of cimetidine, piperonyl butoxide, piperine, diethyldithiocarbamate, chlormethiazole, and any combination thereof.

16. The method of claim 14, wherein said cytochrome P450 is a cytochrome P450 2B family member.

17. The method of claim 14 wherein said method preserves kidney function.

18. The method of claim 17 wherein said kidney function is measured by BUN, plasma creatinine, glomerular filtration rate, or any combination thereof.

19. A method for treating a patient, the method comprising the steps of:
(a) administering a therapeutically effective amount of a composition to a patient, wherein said composition comprises an expression vector comprising a sequence encoding a cytochrome P450 inhibitor, and wherein said patient has a urinary protein excretion value of greater than about 300 mg per twenty-four hour time period.

20. The method of claim 19 wherein said sequence is an antisense sequence of a cytochrome P450 2B family member.

21. The method of claim 20 wherein said antisense sequence is a partial sequence of a cytochrome 2B family member.

22. A method for treating a patient, the method comprising the steps of:
(a) administering a therapeutically effective amount of a composition to a patient, wherein said composition comprises an agent that inhibits a cytochrome P450 superfamily member, and wherein said agent preserves kidney function.

23. The method of claim 22 wherein kidney function is measured by BUN, plasma creatinine level, glomerular filtration rate, or any combination thereof.
